(19) United States
(12) Patent Application Publication
(10) Pub. No.: US 2012/0088237 A1
(43) Pub. Date: Apr. 12, 2012

(54) Engineering a Novel Methylation-Specific Restriction Endonuclease

(75) Inventors: Zhenyu Zhu, Beverly, MA (US); Shengxi Guan, Stoneham, MA (US); Aine Quimby, Newton, NH (US)

(73) Assignee: NEW ENGLAND BIOLABS, INC., Ipswich, MA (US)

(21) Appl. No.: 13/202,851

(22) PCT Filed: Mar. 4, 2010
(86) PCT No.: PCT/US2010/026200

§ 371 Date: Aug. 23, 2011

Related U.S. Application Data
(60) Provisional application No. 61/158,466, filed on Mar. 9, 2009.

(51) Int. Cl. C12Q 1/68; C12N 9/16; C12N 1/21; C12N 15/55; C12N 15/63

(52) U.S. Cl. 435/6.11; 536/23.2; 435/320.1; 435/252.33; 435/196; 435/6.1

(57) ABSTRACT

A restriction endonuclease is provided that has been engineered to have a cleavage specificity for a DNA recognition sequence containing a modified nucleotide. Methods for engineering enzymes to cleave DNA containing modified nucleotides at specific sequences are provided.

### BACKGROUND

In mammalian cells, DNA methylation forms the basis of chromatin structure, which enables cells to form the myriad characteristics necessary for multicellular life from a single immutable sequence of DNA. DNA methylation is essential for normal development and is associated with a number of key processes including genomic imprinting, X-chromosome inactivation, suppression of repetitive elements and carcinogenesis. In adult somatic tissues, DNA methylation typically occurs in a CpG dinucleotide context in the genome; non-CpG methylation is prevalent in embryonic stem cells (Dodge, et al. Gene 289 (1-2): 41-48 (2002); Haines, et al. Developmental Biology 240 (2): 585-598 (2001)).

In plants, cytosines are methylated both symmetrically (CpG or CpNpG) and asymmetrically (CpNpNp), where N is any nucleotide. DNA methylation involves the addition of a methyl group to the 5 position of the cytosine pyrimidine ring or the number 6 nitrogen of the adenine purine ring. This modification can be inherited through cell division. DNA methylation is typically removed during zygote formation and reestablished through successive cell divisions during development.

Restriction-modification (RM) systems are widely present in prokaryotic genomes. They typically consist of restriction endonucleases, which protect the hosts from invading DNA (e.g., bacteriophages) by cleaving DNA at defined sites, and DNA methyltransferases, which protect host DNA from being degraded by methylating the cognate restriction endonuclease sites. Although RM systems are effective at restricting foreign DNA, bacteriophage species can modify their own DNA, thus acquiring resistance to cleavage by most conventional restriction endonucleases. For example, the bacteriophage genomes can be fully cytosine-methylated (Ehrlich et al. Biochim Biophys Acta 395: 109-119 (1975)). The early observation that maintenance of foreign methyltransferase genes in E. coli induces an SOS response led to the discovery of the McrA, McrBC and Mrr systems (Raleigh Mol Microbiol 6: 1079-1086 (1992); and Heitman et al. J Bacteriol 169: 3243-3250 (1987)). Other examples that recognize more specific sites include DpnI (G^{m6}ATC) and GlaI (G^{m5}CG^{m5}C) (Tarasova BMC Mol Biol 9: 7 (2008) and BisI (G^{m5}CNGC) (Tarasova BMC Mol Biol 9: 7 (2008)). The presence of these methylation-dependent restriction endonucleases allows the hosts to defend against bacteriophages with modified DNA. In most cases where restriction endonucleases are capable of cleaving a methylated nucleotide in the recognition sequence, the same restriction endonuclease is also capable of cleaving unmethylated recognition sequences thereby limiting its usefulness as reagents for studying methylation in eukaryotic genomes. As the significance of epigenetics increases, so does the need for simple enzymatic methods for specifically identifying methylated nucleotides.

### SUMMARY

In an embodiment of the invention, an isolated DNA encoding a protein is provided where the DNA has at least 85% or 95% or 95% sequence identity with SEQ ID NO:3 or stringently hybridizes to SEQ ID NO:3. In another embodiment, the isolated DNA encodes a protein which has at least 85% or 90% or 95% sequence identity with SEQ ID NO:4. For example, the isolated DNA may have an alternate amino acid at position 200 in SEQ ID NO:4 which replaces an arginine found in SEQ ID NO:4. More particularly, the alternate amino acid may be a cysteine. A vector containing the isolated DNA described herein and a host cell for expressing the vector is also provided.

In an additional embodiment, an isolated DNA as described above is provided wherein the protein cleaves a DNA substrate containing a methylated cytosine with at least two-fold increased activity compared with the protein comprising SEQ ID NO:4.

In one embodiment of the invention, the protein is a restriction endonuclease. For example, the restriction endonuclease can cleave a DNA substrate containing a methylated cytosine with at least two-fold increased activity compared with the protein comprising SEQ ID NO:4.

In an embodiment of the invention, a method is provided for creating an enzyme for selectively cleaving one or more modified nucleotides in a substrate DNA that includes: (a) selecting a naturally occurring endonuclease having cleavage activity for a unmodified substrate; (b) creating a set of mutants wherein each mutant has one or more mutations at varying positions in the wild type DNA encoding the endonuclease for example wherein the one or more mutations comprise changing an amino acid to an alanine; and (c) identifying a member of the set of mutants that preferentially cleaves a DNA recognition sequence containing one or more modified nucleotides in the substrate DNA with at least two-fold increased activity compared with the unmutated protein under the same reaction conditions.

The method may additionally include selecting one or more members of the set that have been identified as cleaving one or more methylated nucleotides and changing at least one additional amino acid to another amino acid for identifying improvements in activity and specificity.

Examples of naturally occurring endonucleases that can be modified according to the method include: BpmI, BseYI, BsgI, BspCNI, BsrI, BstNI, BtsI, EcoP15I, Hpyl88I, HpyCH4III, PhoI, SfiI, AleI, BbvCI, BfuAI, BsaWI, BsoBI, BsrBI, BspEI, BssSI, DraIII, EarI, EcoRI, MboI, MspI, NciI, NmeAIII, PhoI, SfaNI, StyD4I, TaqI, TliI, XhoI, XmaI, BssAI, AsuII, AjnI, BseBI, BstOI, Bst2UI, BstNI, MvaI, Psp61, PspGI; and isoschizomers and neoschizomers thereof.

In embodiments of the invention, restriction endonucleases of the type described above may be used for analyzing methylation patterns in a eukaryotic genome or for detecting a methylated nucleotide in a DNA. An example of such a restriction endonuclease is a BstNI restriction endonuclease mutant.

### BRIEF DESCRIPTION OF THE DRAWINGS

FIG. 1 provides the gene structure for the BstNI modification system.

FIG. 2 provides the DNA and protein sequences for BstNIM (SEQ ID NOS:1 and 2, respectively).

FIG. 3 provides the DNA and protein sequences for BstNI (SEQ ID NOS:3 and 4, respectively).

FIG. 4 shows the cleavage properties of pACYC184-BstNIM.

FIG. 5 shows an activity assay of the crude lysate from the six colonies obtained after transformation with placzz1, ptaczz1, and pETzz1 vectors.

FIGS. 6A and 6B show the activity of the crude lysate of the selected BstNI clones on lambda DNA (FIG. 6A) and pBC4 DNA (FIG. 6B).

FIGS. 7A and 7B show the activity of the crude lysate of the selected BstNI clones on pBC4 (FIG. 7A) and pBR322 DNA (FIG. 7B).

FIGS. 8A and 8B show a detailed comparison of R200C BstNI (FIG. 8A) and WT BstNI (FIG. 8B) on dcm⁺ and dcm⁻ pBC4.

FIG. 9 shows a summary of cleavage patterns for wild-type and mutant BstNI.

FIG. 10 shows the activity of wild type BstNI on plasmid Litmus 28i. Crude extract of bacteria containing pBAD-BstNI was 10-fold serially diluted and digested the substrate plasmid Litmus 28i for 1 hour at 60′C.

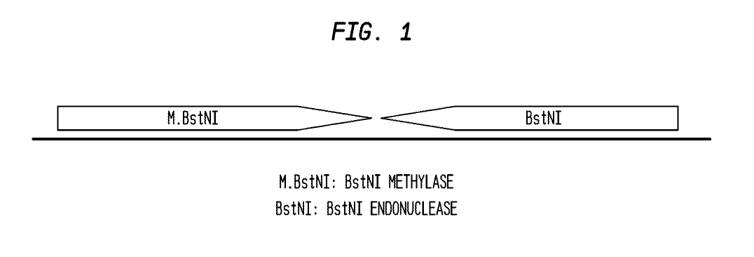

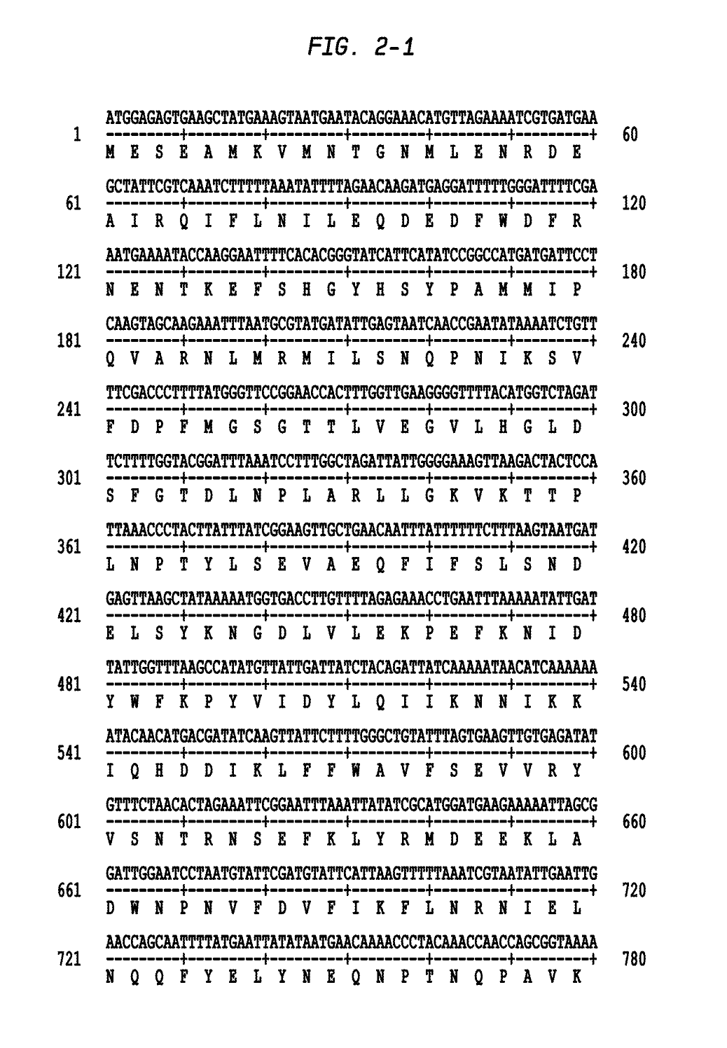

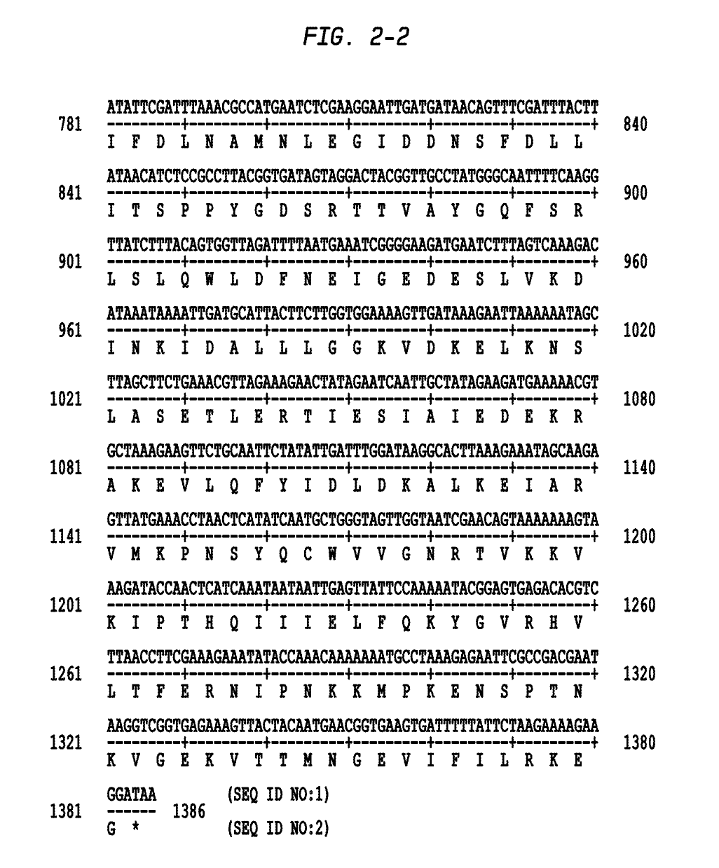

### DETAILED DESCRIPTION OF THE EMBODIMENTS

Methods are provided for increasing the specificity of restriction endonucleases that naturally cleave a specific recognition sequence preferably a sequence containing a cytosine without discriminating between a cytosine that is modified and one that is not. The method relies on identifying mutants of the restriction endonuclease that preferentially cleave a recognition site that contains the modified nucleotide (such as modified cytosine). The product of the methods provided herein may be used in epigenetic analyses.

The term “modified” is intended to include methylated and hydroxymethylated nucleotides.

“Stringent hybridization” is exemplified by the following: 0.75M NaCI, 0.15M Tris, 10 mM EDTA, 0.1% sodium pyrophosphate, 0.1% SLS, 0.03% BSA, 0.03% Ficoll 400, 0.03% PVP and 100 μg/ml boiled calf thymus DNA at 50° C. for about 12 hours and washing 3 times for 30 minutes with 0.1×SET, 0.1% SDS, 0.1% sodium pyrophosphate and 0.1M phosphate buffer at 37° C.-55° C.

Examples of restriction endonucleases that when mutated according to the methods described herein could have preferential cleavage for modified nucleotides at the recognition site in DNA as compared with cleavage of unmodified nucleotides at the same site include restriction endonuclease families represented by BamHI, BcgI, BstYI, BglII, PvuI, AsiSI, BpmI, BseYI, BsgI, BspCNI, BsrI, BstNI, BtsI, EcoP15I, Hpyl88I, HpyCH4III, PhoI, SfiI AIeI, BbvCI, BfuAI, BsaWI, BsoBI, BsrBI, BspEI, BssSI, DraIII, EarI, EcoRI, MboI, MspI, NciI, NmeAIII, PhoI, SfaNI, StyD4I, TaqI, TliI, XhoI, XmaI, BssAI, AsuII; and isochizomers and neoschizomers thereof; EcoRII related endonucleases including AjnI, BseBI, BstOI, Bst2UI, BstNI, MvaI, Psp61 and PspGI, some of which are neoschizomers (see REBASE®, New England Biolabs, Inc. (NEB), Ipswich, Mass.).

In one embodiment, the DNA encoding a restriction endonuclease to be modified is mutated so as to specifically alter one or more amino acids in the expressed protein to a different amino acid such as an alanine. This can be done systematically for example by starting at one end of the amino acid sequence of the protein and progressing through to the other end. Each mutant is assayed for cleavage activity using DNA that contains recognition sequences with and without a modified nucleotide such as two different oligonucleotide substrates or plasmids—one having an unmodified recognition sequence, the other containing a modified recognition sequence. When a particular mutant is identified as causing the restriction endonuclease to have greater specificity for modified sites than the wild type enzyme, this mutant is cloned.

The increase in specificity may be at least 2-fold for example at least 5-fold or at least 10-fold or at least 50-fold or at least 100-fold or at least 500-fold or at least 1000-fold preference for modified versus unmodified cytosine in the DNA substrate.

The mutated amino acid(s) identified above within the protein are then subjected to additional targeted mutations which substitute the mutated amino acid(s) for each of the remaining 18 possible amino acids to obtain the optimum mutation at a particular location for cleavage of methylated amino acids.

The activity of different mutants can be readily ascertained using the method described in Example 1 for BstNI. To determine the effect of a mutation in a restriction endonuclease variant on methylated versus unmethylated substrates, the minimal concentration of enzyme required for complete digestion of unmethylated or methylated substrate was determined. The enzyme can be rapidly obtained for example by lysing transformed cells, spinning down cell debris and utilizing supernatant which can then be serially diluted and tested on a fixed amount of a DNA substrate at a standard temperature and for a standard time. The product of the digestions can then be compared using gel electrophoresis. If the minimal concentration of the mutant enzyme required for complete digestion of a modified substrate is 10-fold less than that for unmethylated substrate, this variant is recorded as favoring methylated substrate over unmethylated substrate by 10-fold.

In one embodiment of the invention, mutations from different clones are combined to enhance the activity of the endonuclease and its preference for modified nucleotides in the recognition sequence.

In an embodiment of the invention, restriction endonucleases are used to analyze methylation patterns in genomic DNA where the analysis relies on specific recognition sequences. A plurality of endonucleases may be used in an analysis wherein the following circumstances arise: (a) at least one endonuclease is specific for a recognition sequence containing at least one modified nucleotide; (b) optionally one or more restriction endonucleases cleave in recognition sequences that may or may not contain the modified nucleotide; and (c) optionally one or more endonucleases only cleave at recognition sequences that do not contain a modified nucleotide. For example, similar DNAs may be digested with BstNI R200C (see below) in parallel with PspGI. The separate cleavage patterns of these enzyme digests may be correlated for epigenetic analyses.

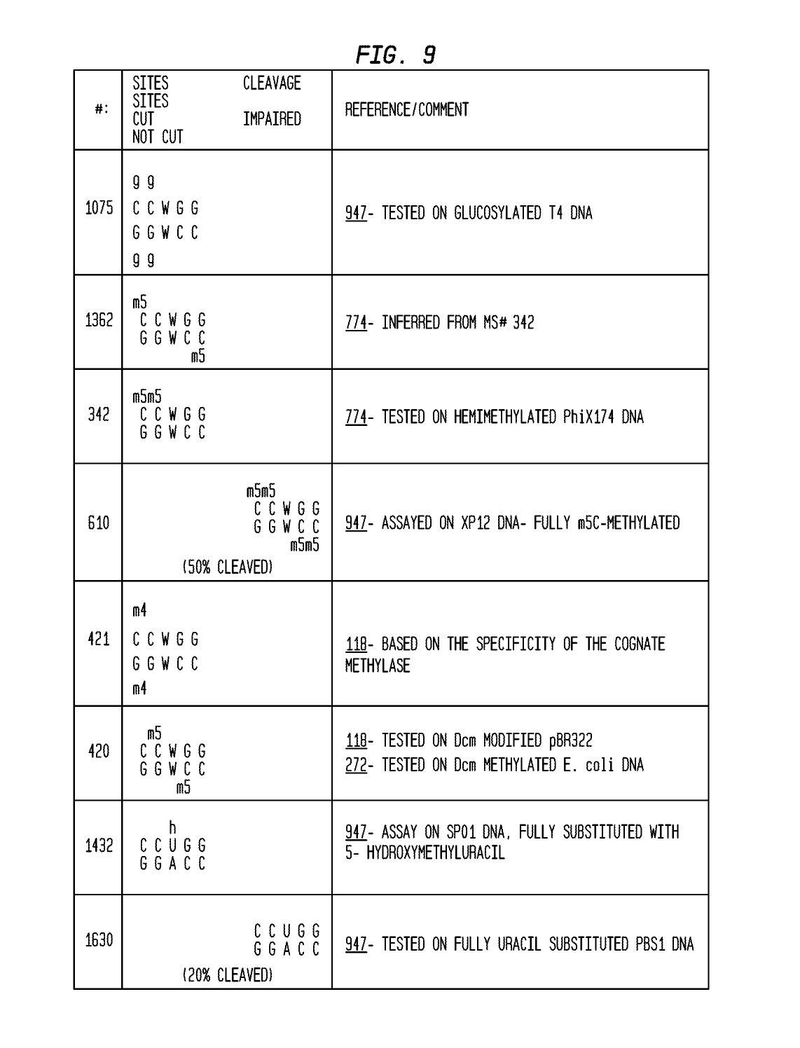

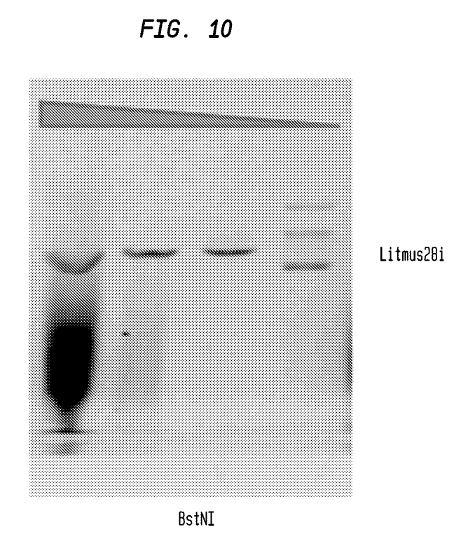

In one embodiment of the invention, wild-type BstNI, which is a Type IIP restriction endonuclease from Bacillus stearothermophilus and recognizes and cuts CC/WGG at both CC/WGG and C^{5m}C/WGG (see FIG. 9) is mutated to preferentially cleave C^{5m}C/WGG. For example, the mutated BstNI may include a mutation at R200 for example R200C.

Details provided in the following examples are not intended to be limiting.

All references cited herein, including U.S. provisional application Ser. No. 61/158,466 filed Mar. 9, 2009, are hereby incorporated by reference.

### EXAMPLES

### Example 1

### Cloning BstNI Restriction Endonuclease which was Previously Only Available as an Isolate from the Native Host

Genome sequencing of the native strain of Bacillus stearothermophilus using shotgun cloning and 454 sequence technology (454 Life Sciences, Branford, Conn.) revealed a sequence which was similar to M.PspGI (GenBank #AF067805) and M.MvaI (GenBank #X16985), both Type IIP restriction endonucleases which recognize CCWGG. It was assumed that this sequence was M.BstNI which methylates the inner cytosine to form C⁴CWGG. Immediately adjacent to the BstNIM gene was an 1224 bp open reading frame. It was hypothesized that this open reading frame encoded BstNI.

(a) Cloning BstNIM

The following primers were used for PCR to amplify the BstNI methylase gene:

(BstNIMF)
(SEQ ID NO: 5)
5′-GGTGGTGGATCCGGAGGTACCTGGATGGAGAGTGAAGCTATGAAA
GTAATGAAT-3′
and
(BstNIMR)
(SEQ ID NO: 6)
5′-GGTGGTGCATGCGCCTGGTTATCCTTCTTTTCTTAGAATAAAAATC
AC-3′.

The PCR reaction mix had the following composition:

10 μl Bacillus stearothermophilus genomic DNA;
2 μl 40 μM primer M.BstNI-F (SEQ ID NO:5);
2 μl 40 μM primer M.BstNI-R (SEQ ID NO:6);
2 μl Vent® DNA polymerase (NEB, Ipswich, Mass.);
4 μl 10 mM dNTP;
10 μl Thermopol buffer (NEB, Ipswich, Mass.); and

### 70 μl H₂O.

The PCR was performed at 94° C. for 5 min, then 30 cycles of 94° C. at 30 sec, 55° C. at 30 sec, 72° C. at 1 min 30 sec, followed by a 1 min 30 sec extension. The PCR product was column-purified and digested with BamHI and SphI, column-purified again, ligated to vector pACYC184, and digested with BamHI, SphI and calf intestinal phosphatase (CIP). The ligated product was then transformed into ER2833, and plated on Luria-Bertani (LB) plate with 33 μg/ml Chloramphenicol (Cam), and incubated at 37° C. overnight.

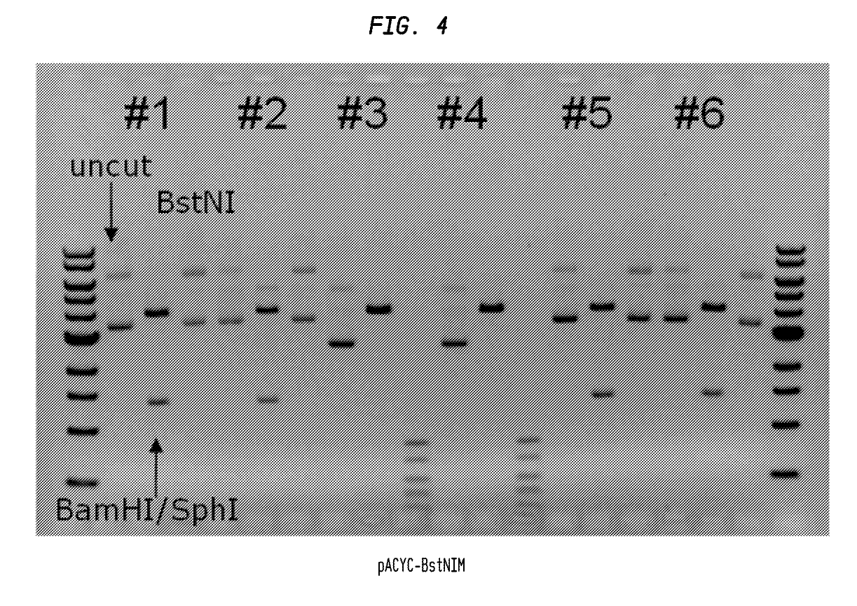

Six colonies were then picked and grown in LB with 33 μg/ml Cam. Plasmids were then extracted and digested with BamHI, SphI and BstNI, separately. Plasmids from colonies #1, 2, 5 and 6 had inserts of the expected size and were resistant to BstNI digestion (FIG. 4). The cells with plasmids resistant to BstNI were re-grown and made chemically competent.

(b) Cloning of BstNI

The following primers were used for PCR to amplify BstNI endonuclease:

(BstNIRF)
(SEQ ID NO: 7)
5′-GGTGGTCTGCAGGGAGGTAAATAAATGGATAAAGAATTAAAAAATTA
TATGGAT-3′
and
(BstNIRR)
(SEQ ID NO: 8)
5′-GGTGGTGGTACCCTATGGTTTTACTAAAATTTGCTGTTCTTT-3′.

The PCR reaction mix had the following composition:

10 μl Bacillus stearothermophilus DNA;
2 μl 40 μM primer BstNIRF (SEQ ID NO:7);
2 μl 40 μM primer BstNIRR (SEQ ID NO:8);
2 μl Vent® DNA polymerase (NEB, Ipswich, Mass.);
4 μl 10 mM dNTP;
10 μl Thermopol buffer (NEB, Ipswich, Mass.); and

### 70 μl H₂O.

The PCR was performed at 94° C. for 5 min, then 30 cycles of 94° C. 30 sec, 55° C. 30 sec, 72° C. 1 min 30 sec, followed by a 1 min 30 sec extension. The PCR product was then column-purified and digested with PstI and Acc65I, column-purified again and ligated to vector placzz1 (a pUC19 derivative with a multiple-cloning site). The ptaczz1 and pETzz1 vectors which are ptac and pET vectors with multiple cloning sites were digested with SbfI, Acc65I and CIP. The ligated product was then transformed into ER2833 with the pACYC184-BstNIM, and plated on LB plates with 100 μg/ml Ampicillin (Amp) and 33 μg/ml Cam, and incubated at 37° C. overnight.

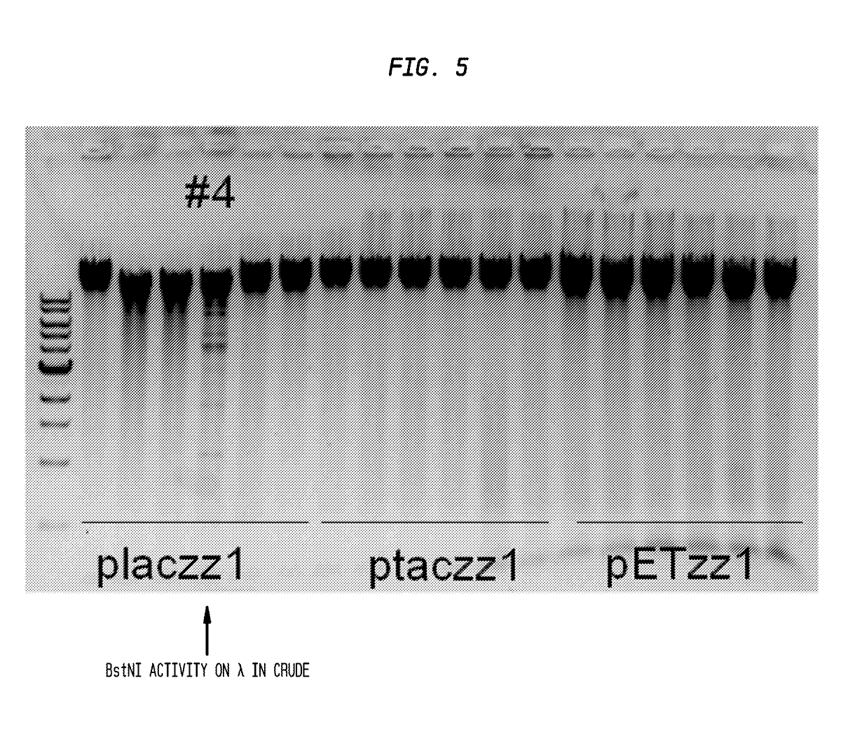

Six colonies from each vector were picked and grown in 3 mL LB with 100 μg/ml Amp and 33 μg/ml Cam at 37° C. 0.5 mM IPTG was added to the final concentration for the induction and the cells were grown for another 16 hours at 37° C. after induction. The cells were then sonicated and activities were tested on the lambda DNA at 60° C. for 1 hour. One colony (#4) showed partial activity on lambda DNA (FIG. 5).

The 1224 bp putative BstNI gene was cloned into placzz1 for transforming a M.BstNI-protected E. coli strain. Several colonies resulting from the transformation were picked in order to screen for BstNI activity. No restriction endonuclease activity was detected. Sequencing these plasmids revealed that the cloned DNA contained a variety of mutations in the BstNI gene sequence when compared with the open reading frame in the genomic sequence. Surprisingly, one clone, which contained an arginine to cysteine mutation, retained its expected recognitionof CCWGG but predominantly cleaved C^{5m}CWGG in contrast with BstNI obtained from a wild type host which recognized and cleaved both methylated and unmethylated CCWGG to a similar extent. The mutated amino acid was identified at position 371 in the protein sequence.

The putative BstNI was then cloned into a more tightly controlled pETzzI vector to avoid potential toxicity of the enzyme. Protein expression was induced by IPTG. After cloning and transformation, again no detectable activity was obtained. Sequencing of the vectors revealed that the DNA encoding the putative BstNI contained a variety of mutations.

Because of difficulties in cloning the active wild type BstNI endonuclease, a different approach was taken. It was decided to investigate whether the genomic open reading frame was wrongly designated and the gene in fact was initiated by an internal ATG start codon. Consequently, a purified preparation of BstNI from the native strain was sequenced at the N-terminal and this was compared with the amino acid sequence encoded by the 1224 bp open reading frame. The N-terminal amino acid sequence was found to be MMDXXKTFIKKLEEIKAKGYIXTL (SEQ ID NO:9). When this amino acid sequence was aligned with the putative BstNI, it was found that translation actually started from an RNA transcribed from the middle of the putative 1224 BstNI gene.

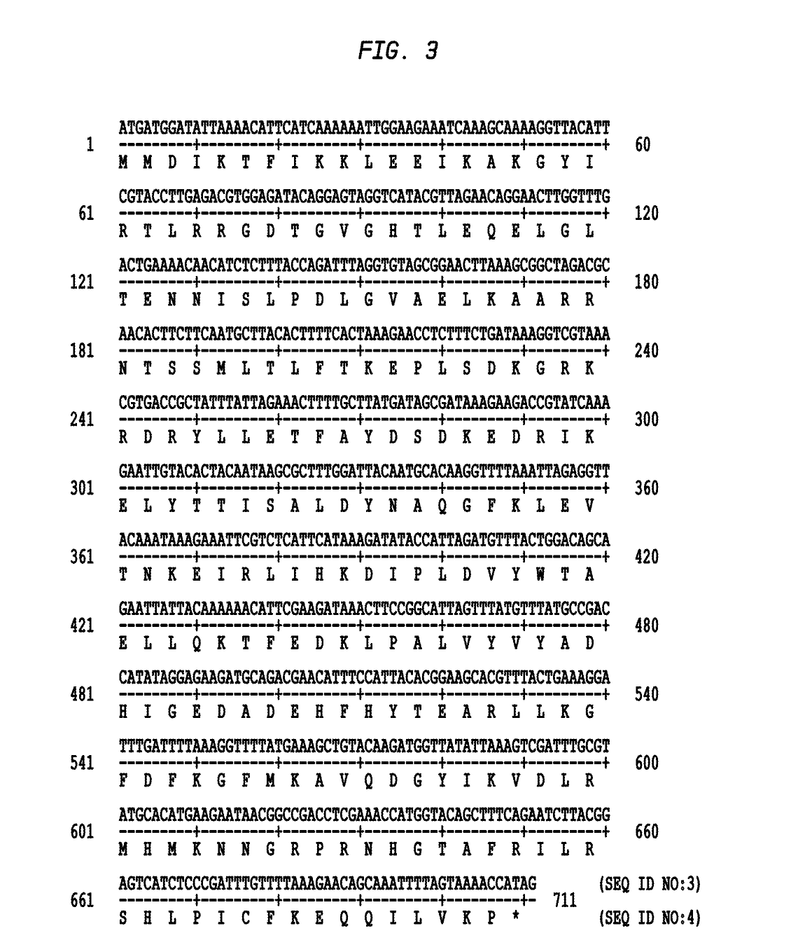

It was concluded that the BstNI gene was actually 711 bp and the BstNI protein contained 236 amino acids (see FIG. 3, SEQ ID NOS:3 and 4, respectively). It was further surmised that toxicity of the restriction endonuclease may have resulted in the absence of detectable endonuclease activity and the appearance of a range of mutations. Consequently, a cloning vector was selected with tight control.

The 711 bp BstNI gene was cloned into the pBAD241 vector, which is a pBAD024 derivative that is tightly controlled by AraC activator and can be induced by arabinose (Guzman et al. J. Bacteriol. 177(14): 4121-4130 (1995)). After cloning, transformation and sequencing, a plasmid containing a single nucleotide deletion (cytosine “C” at position 24) in the BstNI gene was isolated. Inverse PCR mutagenesis was used to add the missing cytosine back into the gene. (This can be done for example using commercially available kits such as QuikChange® provided by Stratagene Inc., now Agilent Technologies, La Jolla, Calif.).

The DpnI-digested PCR products were transformed into a pre-modified E. coli strain (ER2833) containing pACYC-BstNIM. Colonies were picked and grown in LB media with 100 μg/ml Amp and 33 μg/ml Cam. 1 ml out of each 3 ml overnight culture was pelleted and the supernatant was removed. The pellet was resuspended in 50 μl H₂O containing 10 μg/ml RNaseA. This bacterial suspension was sonicated for 6 seconds and incubated in 55° C. for 30 minutes. The remaining cellular debris was pelleted for 10 min at 12,000 rpm.

The supernatant was diluted to 1/10, 1/100 and 1/1000 with H₂O. 3 μl of either original or diluted supernatant containing BstNI variants were added to the following 30 μl digestion reaction system: 3 μl 10×NEB4 buffer (NEB, Ipswich, Mass.), 0.6 μg pBC4(dcm⁺) or pBC4(dcm⁻) and supplementary H₂O. The reaction was incubated at 60° C. for 1 hour. The reaction products were resolved in an agarose gel. To determine the effect of a mutation on BstNI with respect to methylated versus unmethylated pBC4 substrate cleavage activity, the minimal concentration of enzyme required for complete digestion of unmethylated or methylated pBC4 was determined. If the minimal concentration of the BstNI variant required for complete digestion of methylated pBC4 was 10-fold less than that for unmethylated pBC4, it was concluded that this variant favored methylated substrate over unmethylated substrate by 10-fold.

After mutagenesis and transformation of E. coli host cells, a clone was isolated with cleavage activity.

Upon sequencing this plasmid, two additional alterations to the protein sequence were detected. These were: 1) the distance between the Shine Delgarno ribosomal binding site sequence and the start of translation (ATG) was 53 nt long instead of the designed 16 nt long; and 2) the ATG coding the first methionine was missing.

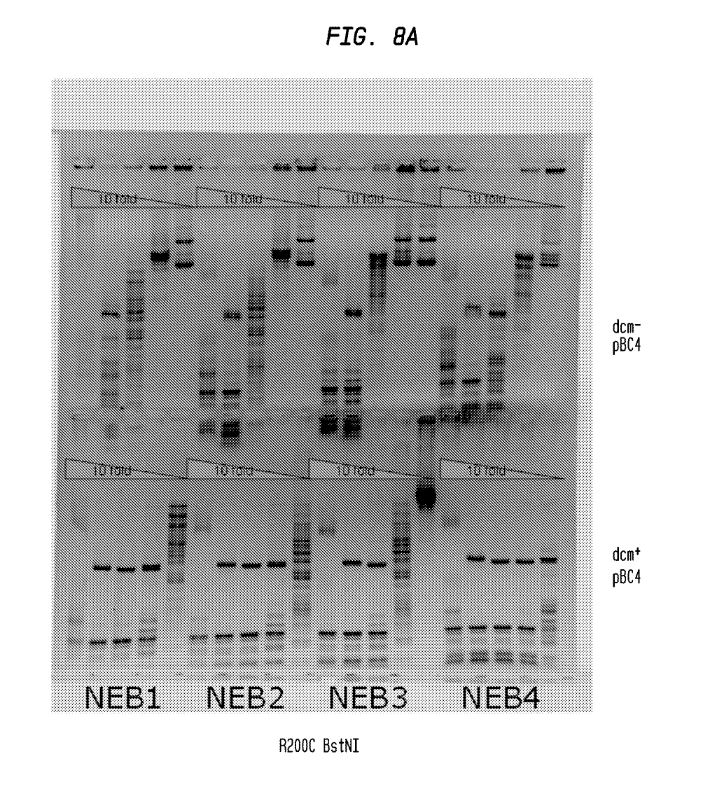

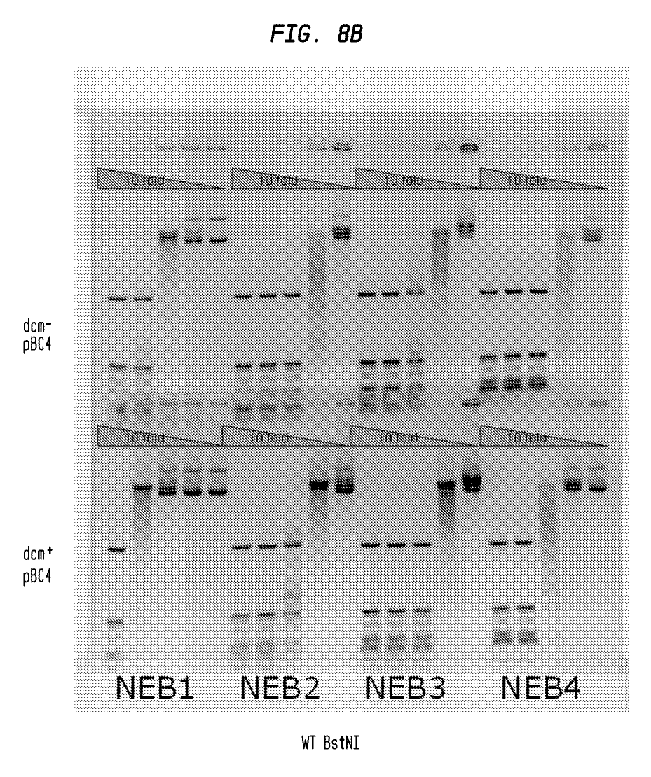

To add back the first methionine, inverse PCR mutagenesis was again used to correct the BstNI gene. After transformation and sequencing, the correct BstNI gene was confirmed in pBAD241 vector. The cells containing the 711 bp fragment were able to produce an active wild type BstNI which characteristically cleaved both methylated and unmethylated DNA (FIG. 8B).

### Example 2

### Obtaining and Characterizing an BstNI Mutant with Increased Specificity for a Recognition Sequence Containing Methylated Cytosine

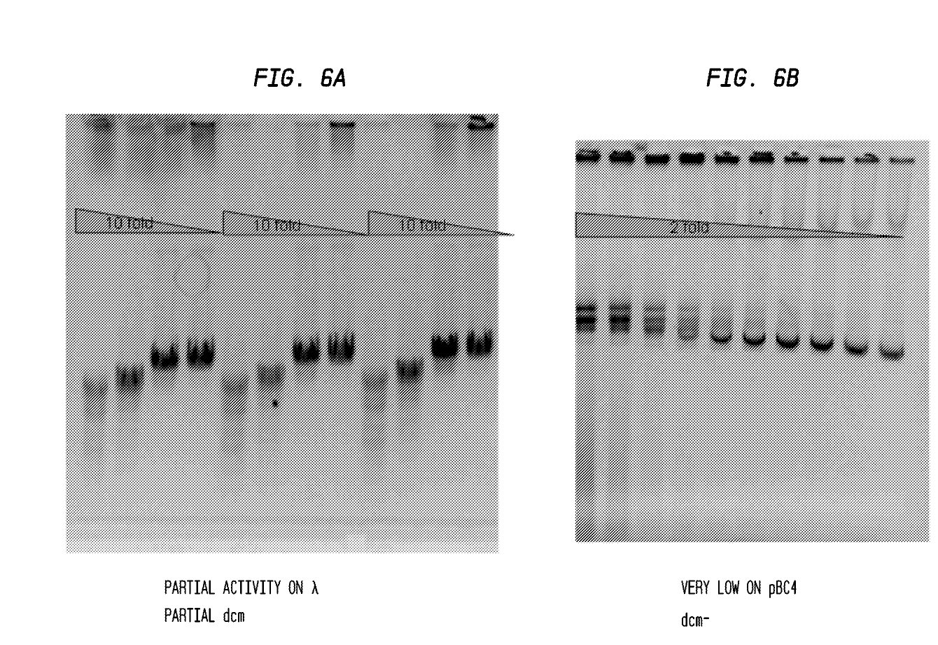

The plasmid extracted from #4 in Example 1 was retransformed to ER2833(pACYC-BstNIM). 3 colonies were picked and regrown as in the above procedure. The cells were sonicated and tested on lambda DNA and a plasmid pBC4 (dam⁻ and dcm⁻). The cell extracts showed partial cleavage activity of lambda DNA and low cleavage activity of pBC4 (FIGS. 6A and 6B).

The DpnI-digested PCR products were transformed into a pre-modified E. coli strain (ER2833) containing pACYC-BstNIM. Colonies were picked and grown in LB media with 100 μg/ml Amp and 33 μg/ml Cam. 1 ml out of each 3 ml overnight culture was pelleted and the supernatant was removed. The pellet was resuspended in 50 μl H₂O containing 10 μg/ml RNaseA. This bacterial suspension was sonicated for 6 seconds and incubated in 55° C. for 30 minutes. The remaining cellular debris was pelleted for 10 min at 12,000 rpm.

The supernatant was diluted to 1/10, 1/100 and 1/1000 with H₂O. 3 μl of either original or diluted supernatant containing BstNI variants were added to the following 30 μl digestion reaction system: 3 μl 10×NEB4 buffer (NEB, Ipswich, Mass.), 0.6 μg pBC4(dcm⁺) or pBC4(dcm⁻) and supplementary H₂O. The reaction was incubated at 60° C. for 1 hour. The reaction products were resolved in an agarose gel. To determine the effect of mutation of BstNI on methylated versus unmethylated pBC4, the minimal concentration of enzyme required for complete digestion of unmethylated or methylated pBC4 was determined. If the minimal concentration of the BstNI variant required for complete digestion of methylated pBC4 was 10-fold less than that for unmethylated pBC4, it was concluded that this variant favored the methylated substrate 10-fold over the unmethylated substrate.

After mutagenesis and transformation of E. coli host cells, a clone was isolated with activity (see FIG. 8A).

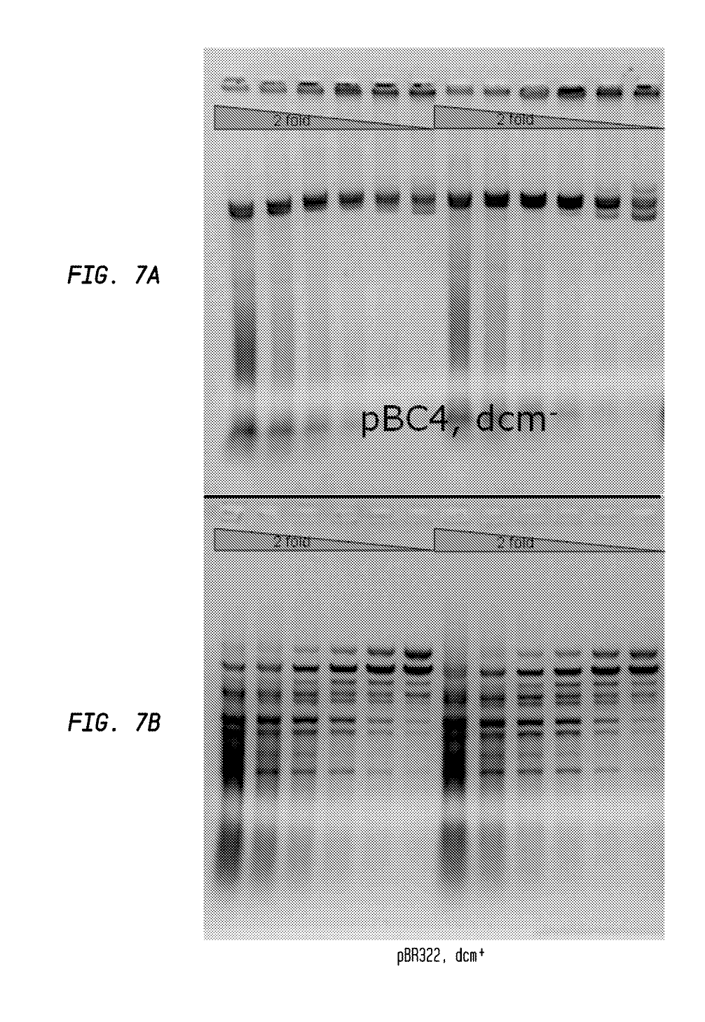

However, when tested on pBR322(dcm⁺) and pBC4(dcm⁻) in parallel, the cell extracts showed much higher activity on pBR322 than pBC4 (FIGS. 7A and 7B). While the cell extract produced a clear banding pattern on the pBR322 observable at 32-fold dilution, there was no clear banding pattern from digestion of the pBC4, confirming the above. Hence, it was concluded that the cell extract containing the mutant BstNI digested only the C^{5m}CWGG and not the unmethylated CCWGG.

The plasmid was sequenced and the BstNI expressed from the 711 bp gene was found to contain a mutation at R200 which was preferentially converted to a cysteine. BstNI R200C displayed a substantially higher ratio of cleavage of methylated/unmethylated substrate than the unmutated BstNI.

Dcm⁻ pBC4 was transformed into a cicm⁺ strain ER2984, and cicm⁺ pBC4 was extracted from this ER2984. A detailed comparison of R200C BstNI and wild-type.BstNI on dcm⁻ and cicm⁺ pBC4 in 4 different NEB buffers was performed (FIGS. 8A and 8B). The results were summarized in Table 1.

TABLE 1
The methylation preferences on (dcm⁺ pBC4)/(dcm⁻ pBC4)

| | R200C BstNI | WT BstNI | R200C BstNI/WT BstNI |
|---|---|---|---|
| NEB1 | 100 | 0.1 | 1000 |
| NEB2 | 100 | 0.5 | 200 |
| NEB3 | 10 | 2 | 5 |
| NEB4 | 100 | 0.1 | 1000 |

In NEB1 and NEB4 (standard buffers available from NEB, Ipswich, Mass.), the methylation preferences of R200C BstNI were enhanced 1000-fold over wild type BstNI.

### Further Enhancement of BstNI Methylated DNA Cleavage Activity

The R200 position in BstNI was further mutated in order to determine whether further improved activity might be achieved by substituting any of the other 18 amino acids at that position. The R200C mutant was identified as optimal.

What is claimed is:

1. An isolated DNA encoding a protein having at least 90% sequence identity with SEQ ID NO:4.

2. An isolated DNA according to claim 1, wherein an arginine at position 200 in SEQ ID NO:4 is replaced with alternate amino acid.

3. An isolated DNA according to claim 2, wherein the alternate amino acid is a cysteine.

4. A vector comprising the DNA of claim 1.

5. A host cell transformed by the vector of claim 4.

6. An isolated DNA according to claim 1, wherein the encoded protein is not SEQ ID NO:4 but cleaves a DNA substrate containing a methylated cytosine with at least two-fold increased activity compared with a protein comprising SEQ ID NO:4.

7. A restriction endonuclease encoded by a DNA according to any of claims 1-3 and 6.

8. A restriction endonuclease according to claim 7, for cleaving a DNA substrate containing a methylated cytosine with at least two-fold increased activity compared with a protein comprising SEQ ID NO:4.

9. A method for creating an enzyme for selectively cleaving one or more modified nucleotides in a substrate DNA; comprising:
selecting a naturally occurring endonuclease having cleavage activity for a unmodified substrate;
creating a set of mutants, each mutant comprising one or more mutations at varying positions in the wild type DNA encoding the endonuclease; and
identifying a member of the set of mutants that preferentially cleaves a DNA recognition sequence containing one or more modified nucleotides in the substrate DNA with at least two-fold increased activity compared with the unmutated protein under the same reaction conditions.

10. The method according to claim 9, wherein the naturally occurring endonuclease is selected from the group consisting of: BpmI, BseYI, BsgI, BspCNI, BsrI, BstNI, BtsI, EcoP15I, Hpy188I, HpyCH4III, PhoI, SfiI, AleI, BbvCI, BfuAI, BsaWI, BsoBI, BsrBI, BspEI, BssSI, DraIII, EarI, EcoRI, MboI, MspI, NciI, NmeAIII, PhoI, SfaNI, StyD4I, TaqI, TliI, XhoI, XmaI, BssAI, AsuII, AjnI, BseBI, BstOI, Bst2UI, BstNI, MvaI, Psp61, PspGI; and isoschizomers and neoschizomers thereof.

11. The method according to claim 9 or 10, wherein the one or more mutations comprise changing an amino acid to an alanine.

12. The method according to claim 9 or 10, further comprising: selecting one or more members of the set that have been identified as cleaving one or more methylated nucleotides and changing at least one additional amino acid to another amino acid for identifying improvements in activity and specificity.

13. Use of a restriction endonuclease according to claim 7, for analyzing methylation patterns in a eukaryotic genome.

14. Use of a restriction endonuclease mutant according to claim 7, for detecting a methylated nucleotide in a DNA.

15. A restriction endonuclease mutant according to claim 7, wherein the restriction endonuclease is a BstNI variant.


SEQUENCE LISTING (9 sequences)

SEQ ID NO: 1 - DNA, 1386 bp, Bacillus stearothermophilus
atggagagtgaagctatgaaagtaatgaatacaggaaacatgttagaaaatcgtgatgaagctattcgtcaaatctttttaaatattttagaacaagatgaggatttttgggattttcgaaatgaaaataccaaggaattttcacacgggtatcattcatatccggccatgatgattcctcaagtagcaagaaatttaatgcgtatgatattgagtaatcaaccgaatataaaatctgttttcgacccttttatgggttccggaaccactttggttgaaggggttttacatggtctagattcttttggtacggatttaaatcctttggctagattattggggaaagttaagactactccattaaaccctacttatttatcggaagttgctgaacaatttattttttctttaagtaatgatgagttaagctataaaaatggtgaccttgttttagagaaacctgaatttaaaaatattgattattggtttaagccatatgttattgattatctacagattatcaaaaataacatcaaaaaaatacaacatgacgatatcaagttattcttttgggctgtatttagtgaagttgtgagatatgtttctaacactagaaattcggaatttaaattatatcgcatggatgaagaaaaattagcggattggaatcctaatgtattcgatgtattcattaagtttttaaatcgtaatattgaattgaaccagcaattttatgaattatataatgaacaaaaccctacaaaccaaccagcggtaaaaatattcgatttaaacgccatgaatctcgaaggaattgatgataacagtttcgatttacttataacatctccgccttacggtgatagtaggactacggttgcctatgggcaattttcaaggttatctttacagtggttagattttaatgaaatcggggaagatgaatctttagtcaaagacataaataaaattgatgcattacttcttggtggaaaagttgataaagaattaaaaaatagcttagcttctgaaacgttagaaagaactatagaatcaattgctatagaagatgaaaaacgtgctaaagaagttctgcaattctatattgatttggataaggcacttaaagaaatagcaagagttatgaaacctaactcatatcaatgctgggtagttggtaatcgaacagtaaaaaaagtaaagataccaactcatcaaataataattgagttattccaaaaatacggagtgagacacgtcttaaccttcgaaagaaatataccaaacaaaaaaatgcctaaagagaattcgccgacgaataaggtcggtgagaaagttactacaatgaacggtgaagtgatttttattctaagaaaagaaggataa

SEQ ID NO: 2 - protein, 461 aa, Bacillus stearothermophilus
MESEAMKVMNTGNMLENRDEAIRQIFLNILEQDEDFWDFRNENTKEFSHGYHSYPAMMIPQVARNLMRMILSNQPNIKSVFDPFMGSGTTLVEGVLHGLDSFGTDLNPLARLLGKVKTTPLNPTYLSEVAEQFIFSLSNDELSYKNGDLVLEKPEFKNIDYWFKPYVIDYLQIIKNNIKKIQHDDIKLFFWAVFSEVVRYVSNTRNSEFKLYRMDEEKLADWNPNVFDVFIKFLNRNIELNQQFYELYNEQNPTNQPAVKIFDLNAMNLEGIDDNSFDLLITSPPYGDSRTTVAYGQFSRLSLQWLDFNEIGEDESLVKDINKIDALLLGGKVDKELKNSLASETLERTIESIAIEDEKRAKEVLQFYIDLDKALKEIARVMKPNSYQCWVVGNRTVKKVKIPTHQIIIELFQKYGVRHVLTFERNIPNKKMPKENSPTNKVGEKVTTMNGEVIFILRKEG

SEQ ID NO: 3 - DNA, 711 bp, Bacillus stearothermophilus
atgatggatattaaaacattcatcaaaaaattggaagaaatcaaagcaaaaggttacattcgtaccttgagacgtggagatacaggagtaggtcatacgttagaacaggaacttggtttgactgaaaacaacatctctttaccagatttaggtgtagcggaacttaaagcggctagacgcaacacttcttcaatgcttacacttttcactaaagaacctctttctgataaaggtcgtaaacgtgaccgctatttattagaaacttttgcttatgatagcgataaagaagaccgtatcaaagaattgtacactacaataagcgctttggattacaatgcacaaggttttaaattagaggttacaaataaagaaattcgtctcattcataaagatataccattagatgtttactggacagcagaattattacaaaaaacattcgaagataaacttccggcattagtttatgtttatgccgaccatataggagaagatgcagacgaacatttccattacacggaagcacgtttactgaaaggatttgattttaaaggttttatgaaagctgtacaagatggttatattaaagtcgatttgcgtatgcacatgaagaataacggccgacctcgaaaccatggtacagctttcagaatcttacggagtcatctcccgatttgttttaaagaacagcaaattttagtaaaaccatag

SEQ ID NO: 4 - protein, 236 aa, Bacillus stearothermophilus
MMDIKTFIKKLEEIKAKGYIRTLRRGDTGVGHTLEQELGLTENNISLPDLGVAELKAARRNTSSMLTLFTKEPLSDKGRKRDRYLLETFAYDSDKEDRIKELYTTISALDYNAQGFKLEVTNKEIRLIHKDIPLDVYWTAELLQKTFEDKLPALVYVYADHIGEDADEHFHYTEARLLKGFDFKGFMKAVQDGYIKVDLRMHMKNNGRPRNHGTAFRILRSHLPICFKEQQILVKP

SEQ ID NO: 5 - DNA, 54 bp, artificial
  primer
ggtggtggatccggaggtacctggatggagagtgaagctatgaaagtaatgaat

SEQ ID NO: 6 - DNA, 48 bp, artificial
  primer
ggtggtgcatgcgcctggttatccttcttttcttagaataaaaatcac

SEQ ID NO: 7 - DNA, 54 bp, artificial
  primer
ggtggtctgcagggaggtaaataaatggataaagaattaaaaaattatatggat

SEQ ID NO: 8 - DNA, 42 bp, artificial
  primer
ggtggtggtaccctatggttttactaaaatttgctgttcttt

SEQ ID NO: 9 - protein, 24 aa, Bacillus stearothermophilus
  misc_feature (4)..(5): Xaa can be any naturally occurring amino acid
MMDXXKTFIKKLEEIKAKGYIXTL